(19) United States
(12) Patent Application Publication
(10) Pub. No.: US 2014/0030716 A1
(43) Pub. Date: Jan. 30, 2014

(54) Novel tumor marker, its monoclonal antibodies and usage

(71) Applicants: Yi-Ming Arthur Chen, Taipei City (TW); Chia-Hung Yen, Taipei City (TW); Kuan-Hsuan Chen, Taipei City (TW)

(72) Inventors: Yi-Ming Arthur Chen, Taipei City (TW); Chia-Hung Yen, Taipei City (TW); Kuan-Hsuan Chen, Taipei City (TW)

(73) Assignee: NATIONAL YANG - MIN UNIVERSITY, Taipei City (TW)

(21) Appl. No.: 13/558,885

(22) Filed: Jul. 26, 2012

(51) Int. Cl. G01N 33/68

(52) U.S. Cl. CPC G01N 33/6893
USPC 435/6.12; 530/350; 530/387.9; 435/7.1; 435/7.92

(57) ABSTRACT

The present invention relates to a novel tumor marker, DEP domain containing 6 (DEPDC6) protein, and its monoclonal antibodies. The present invention further provides the use of DEPDC6 protein and its monoclonal antibodies for the identification and/or treatment of cancer, such as liver cancer.

### FIELD OF THE INVENTION

The present invention relates to a novel tumor marker, DEP domain containing 6 (DEPDC6) protein, and its monoclonal antibodies. The present invention also relates to the use of DEPDC6 protein and its monoclonal antibodies for the identification and/or treatment of cancer, especially to the diagnostic of liver cancer.

### BACKGROUND OF THE INVENTION

Glycine N-methyltransferase (GNMT), also known as a 4S polycyclic aromatic hydrocarbon (PAH) binding protein, has multiple functions. In addition to acting as a major folate binding protein (Yeo E J, et al. Proc Natl Acad Sci USA 1994; 91:210-214), it also regulates the ratio of S-adenosylmethionine (SAM) to S-adenosylhomocysteine (SAH) by catalyzing sarcosine synthesized from glycine (Kerr S J. J Biol Chem 1972; 247:4248-4252). We previously reported that the GNMT is down-regulated in HCC (Liu H H, et al. J Biomed Sci 2003; 10:87-97). Results from a genetic epidemiological study indicate that GNMT is a tumor susceptibility gene for liver cancer (Tseng T L, et al. Cancer Res 2003; 63:647-654). In addition, we reported that GNMT binds benzo(a)pyrene and prevents DNA-adduct formation (Chen S Y, et al. Cancer Res 2004; 64:3617-3623).

We recently reported that GNMT overexpression results in shifts in aflatoxin B₁ (AFB₁) detoxification pathways, reduced AFB₁-DNA adduct formation, and protection against liver carcinogenesis in vivo. We have also reported that both male and female Gnmt −/− mice portray chronic hepatitis and HCC with very high penetrance. However, how GNMT played in liver hepatocarcinogensis remains unclear. To address this issue, we used full length human GNMT as bait in a yeast two-hybrid screen system with a human kidney cDNA library and identified DEP domain containing 6 (DEPDC6) as a GNMT binding proteins.

Peterson et al. recently reported that DEPTOR (DEPDC6) is frequently overexpressed in multiple myelomas, but is low in most cancers including bladder, cervical, prostate, head and beck, thyroid, skin and brain cancer. They identify DEPTOR as an mTOR-interacting protein whose expression is negatively regulated by mTORC1 and mTORC2. Loss of DEPTOR activates S6K1, Akt, and SGK1, promotes cell growth and survival, and activates mTORC1 and mTORC2 kinase activities. DEPTOR overexpression suppresses S6K1 but, by relieving feedback inhibition from mTORC1 to PI3K signaling, activates Akt (see, Peterson T R, et al. Cell. 2009; 137:873-886). However, in that report, the expression results of different organs other than myeloma were collected form microarray database. Also they did not detect the protein expression level of DEPDC6 in other tissues.

mTOR is a highly conserved protein kinase located downstream of PI3K and Akt. Its activation is associated with cell growth, survival, and proliferation. mTOR is found in two functionally and structurally distinct complexes, labeled mTOR complex 1 (mTORC1) and mTOR complex 2 (mTORC2). The dysregulation of mTORC 1 signaling is frequently observed in human cancer tissues. The Wnt signaling pathway plays an important role in embryonic development, regulating cell proliferation and cellular polarity and determining cell fate (Logan, C. and Nusse, R. Annu Rev Cell Dev Biol 2004; 20:781-810). When the Wnt ligand binds to Frizzled receptor its signals are transduced to DVL, followed by the inactivation of β-catenin degradation complexes. The stabilization of β-catenin supports its association with T-cell factor (Tcf) in cell nuclei and activates targeted genes.

The present invention first identified DEP domain containing 6 (DEPDC6) as a GNMT binding proteins, and examined the expression level of DEPDC6 in tumorous tissues from liver and other organs. The present invention provides the use of DEPDC6 in diagnostic of cancerous disease, especially liver cancer.

### SUMMARY OF THE INVENTION

In one aspect, the present invention features a novel tumor marker, DEP domain containing 6 (DEPDC6) protein, which is a GNMT binding protein and comprises the amino acid sequence described in SEQ ID NO: 1 (MEEGGSTGSAGSDSSTSGSGGAQQRELERMAEVLVTGEQLRLRLHEEK VIKDRRHHLKTYPNCFVAKELIDWLIEHKEASDRETAIKLMQKLADRGII HHVCDEHKEFKDVKLFYRFRKDDGTFPLDNEVKAFMRGQRLYEKLMSP ENTLLQPREEEGVKYERTFMASEFLDWLVQEGEATTRKEAEQLCHRLM EHGIIQHVSSKHPFVDSNLLYQFRMNFRRRRRLMELLNEKSPSSQETHDS PFCLGKQSHDNRKSTSFMSVSPSKEIKIVSAVRRSSMSSCGSSGYFSSSPT LSSSPPVLCNPKSVLKRPVTSEELLTPGAPYARKTFTIVGDAVGWGFVVR GSKPCHIQAVDPSGPAAAAGMKVCQFVVSVNGLNVLHVDYRTVSNLIL TGPRTIVMEVMEELEC).

As used in the invention, the term “tumor marker” refers to is a substance that can be elevated in cancer, among other tissue types. An elevated level of a tumor marker may be used to indicate cancer. There are many different tumor markers, each indicative of a particular disease process, and they are used in oncology to help detect the presence of cancer.

According to the present invention, DEPDC6 protein is over-expressed in HCC tumorous tissue and its expression is elevated in colon and breast cancers and the tumor tissues from rectum, kidney and pancreas (see, for example, FIG. 7 and Table 5). It is also proved in the present invention that DEPDC6 protein involves in the Wnt/β-catenin pathway in cell proliferation. Therefore, DEPDC6 protein is characterized as a tumor marker.

In another aspect, this invention features a monoclonal antibody against DEPDC6 protein. In one embodiment, the monoclonal antibody is used in the detection of DEPDC6 protein. In a further embodiment, the monoclonal antibody is used to detect the expression level of DEPDC6 protein in human tissue. In another embodiment, the human tissue is isolated from an organ selected from the group of liver, colon, breast, rectum, kidney, pancreas and stomach.

Further, the present invention relates to a method to detect the presence or expression level of DEPDC6 protein in sample tissue by using a monoclonal antibody against DEPDC6 protein. In a further embodiment, it is provided a kit for detecting the presence or expression level of DEPDC6 protein in sample tissue, which is characterized by comprising a monoclonal antibody against DEPDC6 protein.

Other features or advantages of the present invention will be apparent from the following drawings and detailed description of several embodiments, and also from the appending claims.

### BRIEF DESCRIPTION OF THE DRAWINGS

FIG. 1 shows the characterization of GNMT-DEPDC6 interaction. (a) DEPDC6 domains, depicting prey obtained from a yeast two-hybrid screen. (b) The DEPDC6 carboxyl terminus (including its PDZ domain) was tested for interaction with GNMT. SV40 T antigen combined with p53 served as positive control. Lamin C served as negative control. SD: synthetic dropout medium. (c) Immunoblotting results of co-immunoprecipitation experiments. GNMT was precipitated only in cells expressed HA-DEPDC6, but not in control cells. (d) Indirect immunofluorescence antibody assay for detecting the cellular localization of GNMT and DEPDC6. Yellow represents GNMT-DEPDC6 colocalization (BAR=10 μm). (e) Fluorescence resonance energy transfer-acceptor bleaching (FRET-AB) used to visualize direct protein-protein interactions between GNMT and DEPDC6. FRET was measured using a Leica TCS SP5 Confocal Spectral Microscope. Photobleaching of the GNMT Rhodamine label (white dotted circle) resulted in an increase in DEPDC6 fluorescent signal within the photobleached area demonstrating FRET (BAR=10 μm).

FIG. 2 shows the DEPDC6 PDZ domain that interacts with the GNMT carboxyl terminus (a.a.171-295). Schematics represent (a) DEPDC6, (b) GNMT, and fragments used for mapping interacting domains. DEP1, first DEP domain; DEP2, second DEP domain; N, amino-terminus region of GNMT; C, catalytic domain composed of the separated fragments a.a.37-175 and a.a.243-29524. (c) FRET-AB used to map the interacting domain (see FIG. 1e and Methods section). FRET efficiency was calculated as [(Dpost-Dpre)/Dpost]×100%. The data represented the mean±S.D. (n=5-10).

FIG. 3 shows the diagrams of shows the diagrams of Western blotting for Rabbit antiserum (a) and mouse D5 mAb to HEK293T expressed HA-DEPDC6 (b) reactivity. Anti-HA mAb was used as positive control. Pre: preimmuserum. FIG. 3c shows mAb D5 immunoprecipitated endogenous DEPDC6 from human HCC cells and mouse liver lysates. Lysates form HuH-7 cells and mouse liver were used for IP experiments. Immunocomplex were separated by SDS-PAGE and following by western blot using commercial available anti-DEPDC6 rabbit polyclonal Ab.

FIG. 4 shows that DEPDC6 is frequently over-expressed in HCC tumorous tissue, which enhances transformation potential. (a) Dot plots of DEPDC6 mRNA levels in 30 benign liver tissue samples and 123 paired HCC tumorous (T) and tumor-adjacent non-tumorous tissue samples (TA) (left panel). Each dot represents the relative expression of each sample, normalized to TBP expression. Thick bar on the right side of each group represents mean, whiskers indicate S.D. Middle and right panels represent DEPDC6 expression profiles in T and TA between male and female patients and between patients with or without hepatitis etiology. HBV, patients with HBV sAg (+); HCV, patients with anti-HCV antibody (+); Non, patients with HBV sAg (−) and anti-HCV antibody (−). *, p<0.05; **, p<0.01; ns, non-significant. (b) Immunohistochemical staining of DEPDC6 protein expression in HCC patients. Magnification: 200×. (c) MTT assays (OD540) for quantitating cell growth. Data were normalized against OD540 values on day 1 of each treatment. Growth curve is shown as mean±S.D. td, doubling time. (d) Soft agar assay of HA22T cells were transfected with pHA-DEPDC6 or pcDNA3. Each experiment was performed in triplicate; error bars represent S.D. **, p<0.01.

FIG. 5 is diagrams showing the DEPDC6 participation in Akt/mTOR and Wnt pathways. (a) Neighbor-joining phylogenetic analysis of 11 DEP domains. DH: Dbl-homologous (DH) domain, PH: Pleckstrin homology-like domain. DAX (also call DIX) domain is present in Dishevelled and AXIN. Bootstrap values greater than 700 from 1,000 replicate trees were considered significant (indicated at the nodes of corresponding branches). (b) Co-immunoprecipitation experiments of cell lysates from HEK293T cells transfected with pHA-DEPDC6 for testing the interaction of DEPDC6 with mTOR. (c) Phosphorylation of ribosomal protein S6. Phospho-Akt^{S473}, phosphor-ribosomal protein S6^{S235/236}, and eIF4E (as protein loading control) were detected using PathScan® Multiplex Western Cocktail I (cell signaling). (d) T-cell factor/lymphoid enhancer factor (Tcf/Lef) luciferase reporter assay for determining the effects of DEPDC6 on the Wnt/β-catenin pathway. Experiments were performed in triplicate; error bars represent S.D. (e) T-cell factor/lymphoid enhancer factor (Tcf/Lef) luciferase reporter assay for determining the effects of DEPDC6 on the Wnt/β-catenin pathway when the pathway was activated via β-catenin over-expression.

FIG. 6 shows a model depicting DEPDC6 and GNMT roles in Akt/mTOR and Wnt/β-catenin pathways.

FIG. 7 shows the tissue distribution of DEPDC6. Monoclonal Ab-D5A was used to detect expression profile of DEPDC6 in different organs. Magnification: 200×.

### DETAILED DESCRIPTION OF THE INVENTION

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### EXAMPLE

### Example 1

### Characterization of GNMT-DEPDC6 Interaction

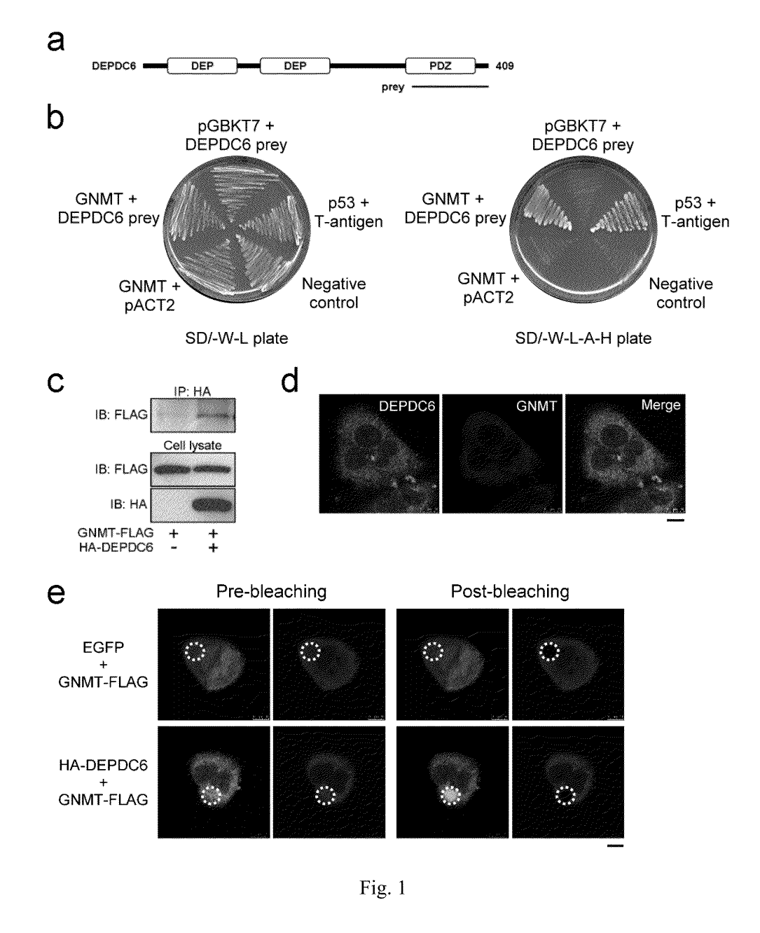

To identify proteins that interact with GNMT, we used full length human GNMT as bait in a yeast two-hybrid screen system with a human kidney cDNA library. Human GNMT cDNA was subcloned into the pGBKT7 vector. A human kidney cDNA library fused to a pACT2 vector served as prey. The DEPDC6 carboxyl terminus (including its PDZ domain) was tested for interaction with GNMT. SV40 T antigen combined with p53 served as positive control. Yeast was grown in media lacking tryptophan and leucine (-W-L), which selects for the presence of plasmids. Only those combinations displaying a strong interaction grew under stringent conditions (i.e., media lacking tryptophan, leucine, adenine and histidine, or -W-L-A-H). According the results, the carboxyl-terminal region of a novel protein named DEP domain containing 6 (DEPDC6) was isolated (FIG. 1a, b). The DEPDC6 gene encodes a 409 amino-acid protein containing two DEP (Dishevelled/Eg1-10/Pleckstrin) domains and one PDZ (Postsynaptic density 95/Discs large/Zonula occludens-1) domain (as shown in FIG. 1a). Their interaction specificity was demonstrated by yeast growth in restrictive media lacking adenine, histidine, leucine, and tryptophan (FIG. 1b).

Interactions of DEPDC6 with GNMT were also confirmed by co-immunoprecipitation (FIG. 1c), an indirect immunofluorescence antibody assay (FIG. 1d), and fluorescence resonance energy transfer-acceptor bleaching (FRET-AB) (FIG. 1e). For the co-immunoprecipitation assay, HEK293T cells were transfected with the indicated plasmids and harvested for co-immunoprecipitation experiments. Cell lysates were incubated with anti-HA antibody and precipitated using protein A/G beads. Immunoprecipitants were resolved on SDS-polyacrylamide gels and analyzed by immunoblotting. GNMT was precipitated only in cells expressed HA-DEPDC6, but not in control cells. In indirect immunofluorescence antibody assay, HuH-7 cells were co-transfected with pHA-DEPDC6 and pGNMT-Flag. Rabbit antiserum against GNMT (produced as described in Example 2) and mouse monoclonal anti-HA antibodies were used to detect the cellular localization of GNMT and DEPDC6, respectively. Goat anti-rabbit IgG conjugated with FITC and goat anti-mouse IgG conjugated with rhodamin were used as secondary antibodies for GNMT and DEPDC6, respectively. (e) Fluorescence resonance energy transfer-acceptor bleaching (FRET-AB) was used to visualize direct protein-protein interactions between GNMT and DEPDC6. pHA-DEPDC6 and pGNMT-FLAG were cotransfected into HuH-7 cells, which were fixed and immunofluorescently stained for DEPDC6 (FITC, energy donor) and GNMT (Rhodamin, energy acceptor). FRET was measured using a Leica TCS SP5 Confocal Spectral Microscope. Photobleaching of the GNMT Rhodamine label (white dotted circle) resulted in an increase in DEPDC6 fluorescent signal within the photobleached area demonstrating FRET. Compared to the pre-bleaching panel, the intensity of the FITC green fluorescence in the HA-tagged DEPDC6 panel of FIG. 1e increased significantly after the rhodamin fluorophore was destroyed by laser-photobleaching.

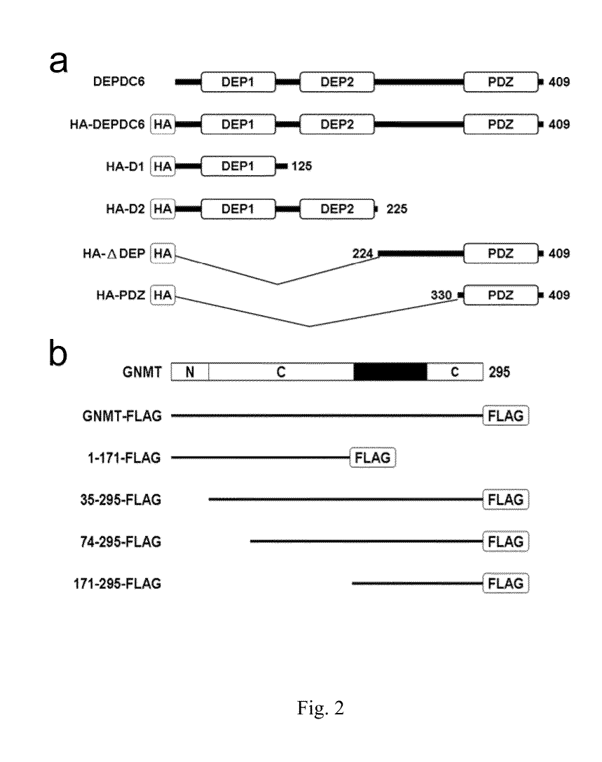

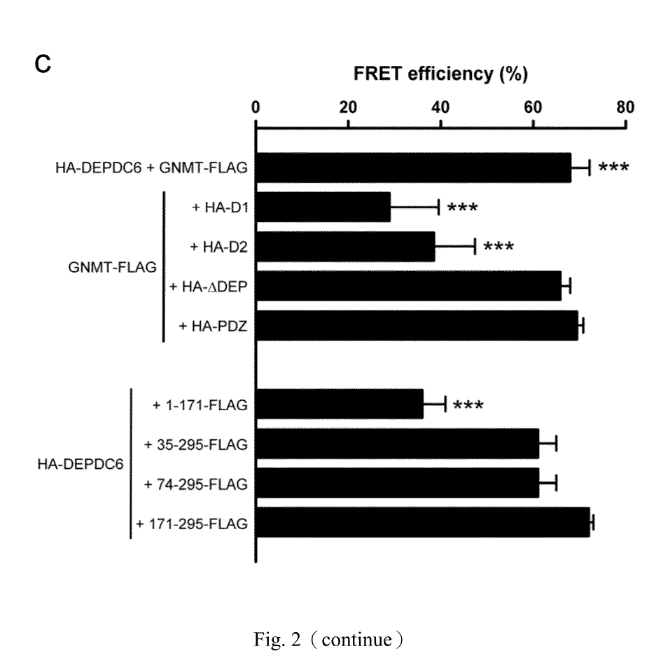

We detected FRET between GNMT and DEPDC6 (n=10, 67%±4% efficiency), but not between GNMT and the EGFP control (i.e., zero FRET efficiency). We constructed eight plasmids containing different domains of either DEPDC6 or GNMT to determine specific domains responsible for their interactions using FRET-AB technique (FIG. 2a, b). As shown in FIG. 2c, FRET efficiency decreased significantly when plasmids containing full-length GNMT were co-transfected with plasmids containing only one DEP domain (HA-D1) or two DEP domains (HA-D2) (p<0.001). In contrast, the FRET efficiency of full-length GNMT and the PDZ domain of DEPDC6 was compatible to that observed between GNMT and DEPDC6. This data is consistent with results from yeast two-hybrid screening. Compare these results with a greater than 50% decrease in FRET efficiency for interactions between full-length DEPDC6 and GNMT lacking the C-terminal 172-295 amino acid fragment (FIG. 2c, p<0.001). The FRET efficiency between DEPDC6 and the C-terminal 171-295 amino acid fragment of GNMT was slightly higher than that between GNMT and DEPDC6, suggesting that interaction between the C-terminal half (amino acid residues 171-295) of GNMT and the PDZ domain of DEPDC6.

### Example 2

### Preparation of Monoclonal Antibodies Against DEPDC6

To generate rabbit anti-DEPDC6 antiserum and monoclonal antibodies against DEPDC6, purified GST-DEPDC6 or His-DEPDC6 were mixed with Freund's complete (for the initial immunization) or incomplete (for the booster injections) adjuvant (Sigma Co., St. Louis, Mo., USA) and the resultant mixture was used as an immunogen. His-DEPDC6 RP was used as a screened antigen for antibody arose by GST-DEPDC6 RP, and vice versa.

Mouse mAbs were produced by hybridoma technique (Liu et al., J Biomed Sci 2003; 10:87-97). The hybridomas were dispensed into six 96-well plates and cultured in a HAT medium (Chu et al., Hybridoma 1993; 12:417-417). The culture supernatants were screened using enzyme immunoassay (EIA) with GST-DEPDC6 RP and His-DEPDC6 RP. Hybridoma cells with high optic density were confirmed with Western blot assay immediately. Each well of cells with positive results were subcloned into a 96-well plate with a cell density of 0.5 cell per well. The resultant single clone with positive results were inoculated at a dosage of 5×10⁶ to a BALB/c mouse which has been primed with 0.5 ml pristine (Sigma-Aldrich) previously. Monoclonal antibodies are purified from the mouse ascites with protein-A antibody purification kits (Pro-Chem Inc. Acton, Mass.) and concentrated using Centricon Plus-80 columns (Millipore). The isotype of each mAb was determined using a commercial kit (SouthernBiotech, Birmingham, Ala.).

High-tittered mAb purified from ascites were diluted with 0.1M NaHCO₃ (pH 8.6) to a concentration of 100 μg/ml, and added to 6 ml sterile polystyrene Petri dishes. After coating overnight at 4° C. in a humidified container, the plates were blocked with the blocking buffer (0.1 M NaHCO₃ pH 8.6, 5 mg/ml BSA, 0.02% NaN3, with a sterilized filter, stored at 4° C.) and incubated for at least 1 hour at 4° C. M13 phages displaying random heptapeptides at the N-terminus of its minor coat protein (pIII) were subsequently added (Ph.D.-7TM Phage Display Peptide Library, New England Biolabs Inc.). The phages bound to the plates were selected and repeatedly screened for 3 times before they were subjected to DNA sequencing. Detailed procedures have been published previously (Cortese et al., Curr Opin Biotechnol. 1996; 7:616-621). The hybridoma producing the mAb-D5 of the present invention was deposited with the German Collection of Microorganisms and Cell Cultures, on Jul. 4, 2012, as Deposit No. DSM ACC3180.

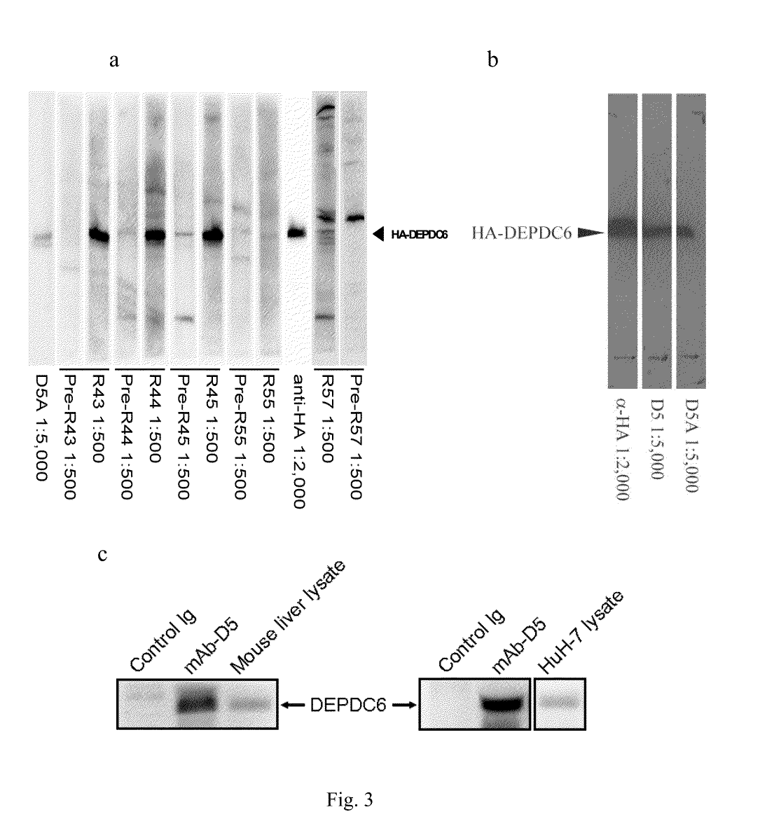

As shown in FIGS. 3a and 3b, the rabbit antiserum-R43 showed a good reactivity against HA-DEPDC6 expressed in HEK293T cell and low background. In addition, mAb-D5 also showed a similar reactivity and background. The epitopes that could be recognized by mAb D5 was mapped using a peptide library displayed on M13 phages. After 3 biopannings, 8 reactive phage colonies were isolated for mAb D5.

Next, we tested whether the mAb D5 can be used in immunoprecipitation (IP) experiments. Mouse liver lysates and HuH-7 cell lysates were used for IP experiment. Lysates were incubated with 10 μg of mAb D5 for 1 h at 4° C., followed by the addition of 20 μl protein-A/G sepharose (Calbiochem, Merck KGaA, Darmstadt, Germany) and incubation for 4 h. The beads were washed three times with lysis buffer and resuspended in sample buffer for SDS-PAGE and Western blot analyses. The result showed that mAb D5 can precipitate endogenous DEPDC6 from both human HCC cell lysate and mouse liver lysate (FIG. 3c).

As shown in Table 1, the heptapeptides displayed at the N-terminus of the phage pIII protein were deduced by sequencing. More than three of isolated phages displayed a major sequence (consensus sequence) that could be used to identify the reactive epitopes for D5. When the consensus sequences were aligned with the human DEPDC6 protein sequence, the best matched regions spanned amino acid residues 245-251 (Table 1).

TABLE 1
epitope mapping of mAb D5

| DEPDC6 | | | | | | | | |
|---|---|---|---|---|---|---|---|---|
| sequence | | ²⁴⁵⁻P | F | C | L | R | K | Q⁻²⁵¹ |
| M13 phage display | #1 | S | H | T | I | — | M | L |
| | #2 | S | W | D | — | A | — | — |
| | #3 | A | —^{a} | — | — | — | V | |
| | #4 | Q | E | * | | | | |
| | #5 | Q | — | M | — | S | L | — |
| | #6 | S | W | D | — | A | — | — |
| | #7 | E | W | D | — | S | L | — |
| | #8 | E | — | R | W | — | S | |
| Consensus sequence | | S | F/W | D | L | R | X | Q |

^{a}residues appeared as conservative amino acids were indicated as dash marks

### Example 3

### DEPDC6 is Frequently Overexpressed in HCC Tumorous Tissue, Which Enhances Transformation Potential

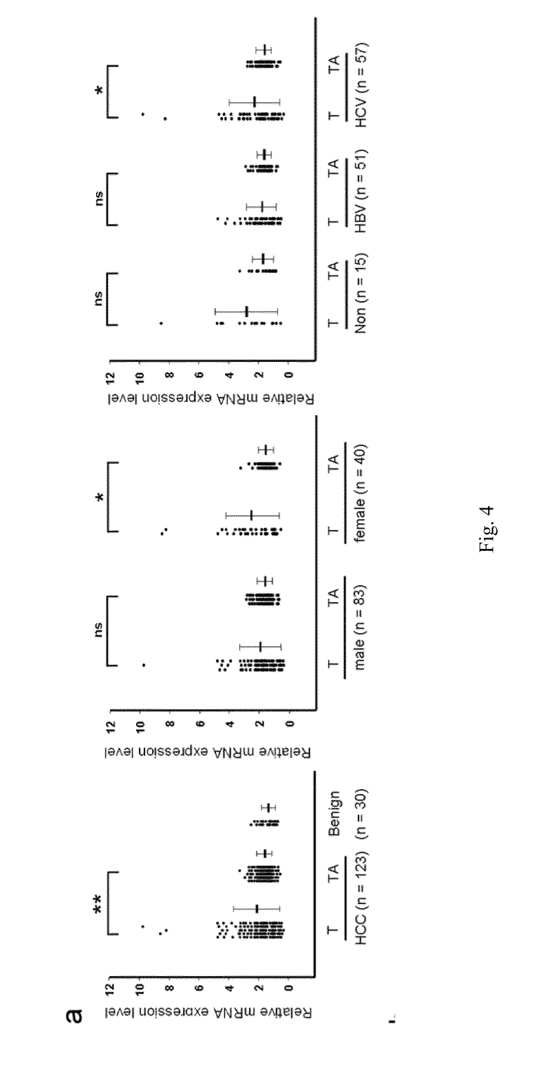

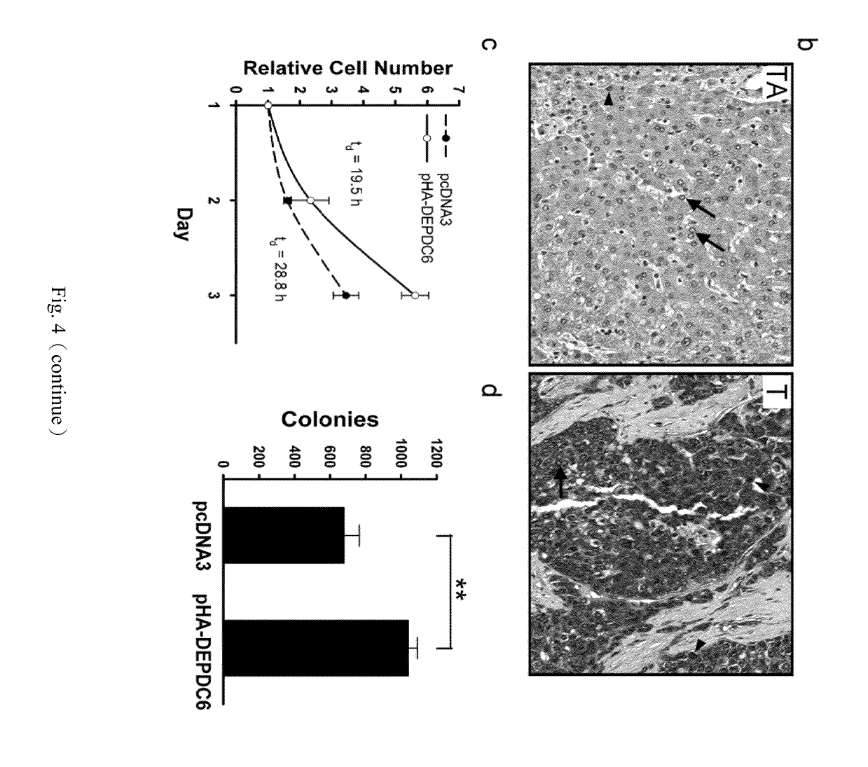

To determine DEPDC6 and GNMT expression levels in different HCC clinical stages or subgroups, we used real-time PCR to analyze 123 paired tumor (T) and tumor-adjacent (TA) tissues from HCC patients (Table 2). Compared to the TA tissues, the DEPDC6 expression level was significantly higher in the tumor tissues (p=0.008) (FIG. 4a). After adjusting the viral hepatitis and liver cirrhosis factors, DEPDC6 expression levels in the tumor tissues of female HCC patients were significantly higher than in the TA tissues (FIG. 4a). This phenomenon was also observed in patients with anti-HCV antibodies and in patients without liver cirrhosis (FIG. 4a and Table 2).

TABLE 2
Demographic data for TLCN specimen donors

| N = 123 | N (%) |
|---|---|
| Age | 60.6 (13-86) |
| Gender | |
| male | 83 (67.5) |
| female | 40 (32.5) |
| Viral infection* | |
| Non | 15 (12.2) |
| HBV | 51 (41.5) |
| HCV | 57 (46.3) |
| Cirrhosis | |
| − | 51 (41.5) |
| + | 72 (58.5) |
| TNM stage | |
| I | 59 (48.0) |
| II | 38 (30.9) |
| IIIA | 24 (19.5) |
| IIIB | 2 (1.4) |

*HBV, HBV sAg (+); HCV, anti-HCV antibody (+); Non, HBV sAg (−) and anti-HCV antibodies (−).

Also note that DEPDC6 expression levels in tumorous tissues from patients with TNM stage I (but not in patients beyond TNM stage I) were significantly higher than those in the TA tissues (Table 3). This may be a result from the elevated DEPDC6 expression level in TA tissues observed in late stage patients. In contrast, GNMT expression levels in tumorous tissues were significantly lower than in TA tissues (p<0.001) (Table 3).

TABLE 3
Clinicopathological associations of DEPDC6 mRNA expression level in HCC patients

| | | Relative mRNA expression level | | | | | |
|---|---|---|---|---|---|---|---|
| Clinical | | GNMT | | | DEPDC6 | | |
| features | | T | TA | | T | TA | |
| | N | mean ± SD (min-max) | mean ± SD (min-max) | p value | mean ± SD (min-max) | mean ± SD (min-max) | p value |
| Overall | 123 | 11.0 ± 18.4 (0.1-109.5) | 33.3 ± 21.5 (0.6-107.3) | <0.001 | 2.1 ± 1.5 (0.3-9.8) | 1.6 ± 0.5 (0.6-3.3) | 0.008 |
| Gender | | | | | | | |
| Male | 83 | 10.1 ± 17.8 (0.1-109.5) | 35.8 ± 21.4 (1.2-93.8) | <0.001 | 1.9 ± 1.4 (0.3-9.8) | 1.6 ± 0.5 (0.7-2.9) | 0.18 |
| Female | 40 | 12.9 ± 19.7 (0.2-77.3) | 28.0 ± 20.9 (0.6-107.3) | 0.001 | 2.5 ± 1.8 (0.5-8.5) | 1.6 ± 0.5 (0.6-3.3) | 0.01 |
| Viral | | | | | | | |
| infection* | | | | | | | |
| Non | 15 | 7.1 ± 8.1 (0.9-24.7) | 47.2 ± 28.8 (14.2-107.3) | 0.001 | 2.8 ± 2.1 (0.5-8.5) | 1.7 ± 0.7 (0.8-3.3) | 0.211 |
| HBV | 51 | 7.6 ± 13.0 (0.1-75.0) | 37.3 ± 18.4 (5.0-93.8) | <0.001 | 1.8 ± 1.0 (0.5-4.7) | 1.6 ± 0.5 (0.7-2.9) | 0.363 |
| HCV | 57 | 15.1 ± 23.2 (0.1-109.5) | 26.0 ± 19.3 (0.6-92.3) | <0.001 | 2.2 ± 1.7 (0.3-9.8) | 1.6 ± 0.5 (0.6-2.7) | 0.016 |
| Cirrhosis | | | | | | | |
| − | 51 | 11.1 ± 16.8 (0.1-80.0) | 38.8 ± 22.2 (0.6-107.3) | <0.001 | 2.6 ± 1.8 (0.4-9.8) | 1.6 ± 0.6 (0.6-3.3) | <0.001 |
| + | 72 | 11.0 ± 19.6 (0.1-109.5) | 29.3 ± 20.1 (1.2-93.8) | <0.001 | 1.8 ± 1.3 (0.3-8.2) | 1.6 ± 0.5 (0.7-2.7) | 0.864 |
| Pathological | | | | | | | |
| stage** | | | | | | | |
| Early | 59 | 14.2 ± 19.4 (0.1-80.0) | 35.0 ± 22.6 (0.6-93.8) | <0.001 | 2.0 ± 1.4 (0.3-8.2) | 1.5 ± 0.5 (0.6-2.5) | 0.022 |
| Late | 64 | 8.1 ± 17.0 (0.1-109.5) | 31.7 ± 20.4 (1.2-107.3) | <0.001 | 2.2 ± 1.6 (0.5-9.8) | 1.8 ± 0.5 (0.8-3.3) | 0.175 |

*HBV, patients with HBV sAg (+); HCV, patients with anti-HCV antibody (+); Non, patients with HBV sAg (−) and anti-HCV antibody (−).
**Early stage, patients with TNM stage = I; Late stage, patients beyond TNM stage I (TNM stage = II + IIIA + IIIB)

Diminished GNMT expression in HCC tumor tissues was consistently found in various patient sub-groups. An anti-DEPDC6 monoclonal antibody (D5) was used in immunohistochemical staining. Among the 51 pairs of tumorous and TA samples that were tested, 88% had either higher or equal expression levels of DEPDC6 in tumorous tissues compared to TA tissues (Table 4).

Slides of HCC patient tissues were incubated with monoclonal antibodies against DEPDC6. As shown in FIG. 4b, Tumor cells (T) showed strong positive signal for DEPDC6 with perinuclear membrane (arrow), cytoplasma, or nucleus (arrowhead) staining, compared to reduced DEPDC6 staining intensity in adjacent non-tumor liver tissues (TA), concluding that DEPDC6 was primarily expressed in HCC cell cytoplasm and especially concentrated in the perinuclear region (FIG. 4b).

Since DEPDC6 is frequently up-regulated in human HCC, we tried to determine if its overexpression enhances the transformational potential of cells. First, we compared the growth of HEK293T cells transfected with DEPDC6-expressing plasmid DNA with those transfected with a vector plasmid, and recorded cell doubling times of 19.5 h and 28.8 h, respectively (FIG. 4c). Furthermore, HA22T cells were transfected with pHA-DEPDC6 or pcDNA3 vector for 24 h, plated in soft agar, and incubated at 37° C. for 28 days. Formed colonies were stained with crystal violet. Results from the soft agar assay shown in FIG. 4d indicate that the transient expression of DEPDC6 in an HCC cell line (HA22T) enhanced colony formation efficiency by more than 50% (p<0.05).

TABLE 4
Immunohistochemical staining of DEPDC6 in human HCC tissues

| | T < TA | T = TA | T > TA | |
|---|---|---|---|---|
| N = 51 | N (%) | N (%) | N (%) | p value |
| All | 6 (11.8) | 34 (66.7) | 11 (21.6) | |
| Gender | | | | 0.91 |
| Female | 3 (11.5) | 18 (69.2) | 5 (19.2) | |
| Male | 3 (12.0) | 16 (64.0) | 6 (24.0) | |
| Viral infection* | | | | 0.034 |
| Non | 5 (29.4) | 11 (64.7) | 1 (5.9) | |
| HBV | 0 (0.0) | 12 (75.0) | 4 (25.0) | |
| HCV | 1 (5.6) | 11 (61.1) | 6 (33.3) | |
| Cirrhosis | | | | 0.106 |
| − | 5 (22.7) | 13 (59.1) | 4 (18.2) | |
| + | 1 (3.4) | 21 (72.4) | 7 (24.1) | |
| Pathological stage** | | | | 0.082 |
| Early | 0 (0.0) | 14 (70.0) | 6 (30.0) | |
| Late | 6 (19.4) | 20 (64.5) | 5 (16.1) | |

*HBV, HBV sAg (+); HCV, anti-HCV antibody (+); Non, HBV sAg (−) and anti-HCV antibodies (−).
**Early stage, TNM stage patients = I; Late stage, patients beyond TNM stage I (TNM stage = II + IIIA + IIIB)

### Example 4

### DEPDC6 Participation in Akt/mTOR and Wnt Pathways

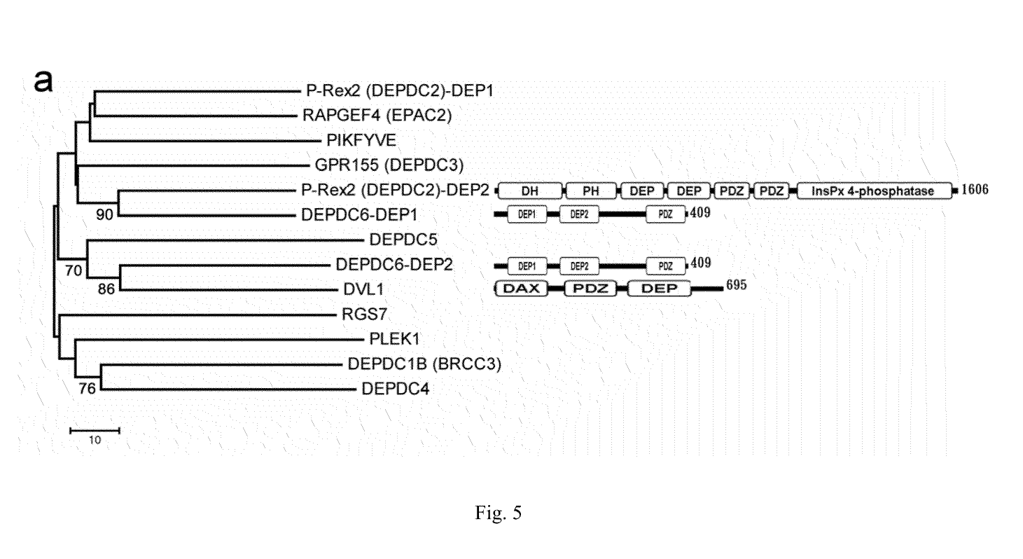

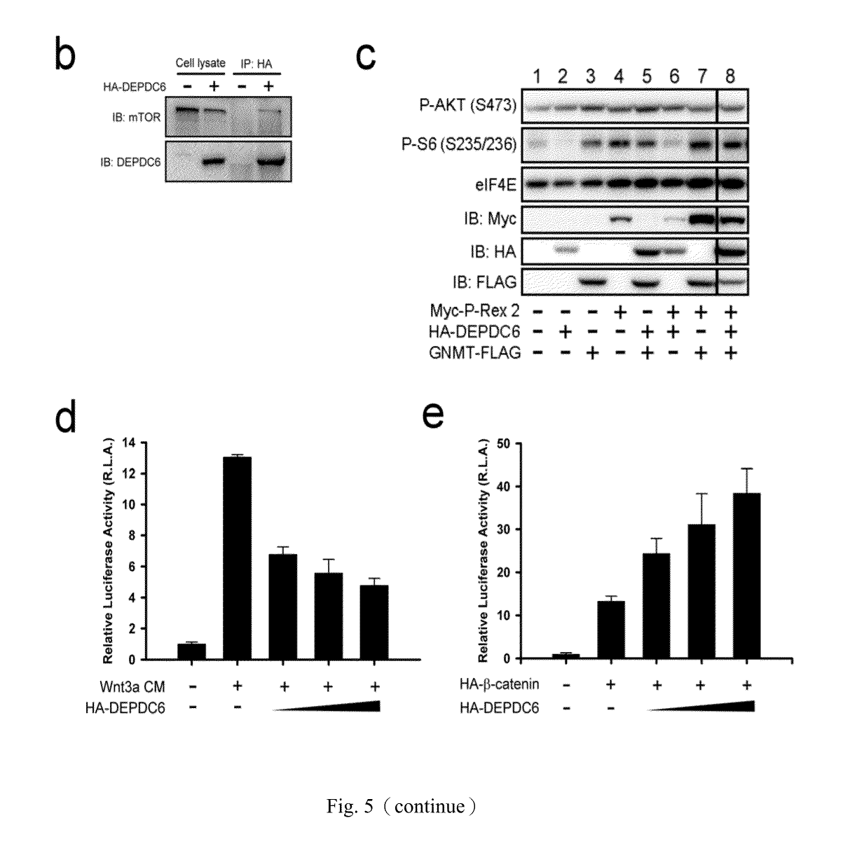

To elucidate possible DEPDC6 functions, we used phylogenetic tree analysis with a neighbor-joining program to study evolutionary relationships among DEP domains from 11 proteins. DNA sequences from the following DEP domains were used to perform neighbor-joining phylogenetic analyses: DEPDC6 (GI: 189571663), P-Rex2 (also known as DEPDC2, GI: 47578114), PIKFYVE (GI: 121583482), RAPGEF4 (also known as EPAC2, GI: 155030205), GPR155 (also known as DEPDC3, GI: 74315999), DEPDC5 (GI: 55749916), DVL1 (GI: 32479520), RGS7 (GI: 156627562), PLEK1 (GI: 156616272), DEPDC1B (also known as BRCC3, GI: 23510330), and DEPDC4 (GI: 90855782). Bootstrap values greater than 700 from 1,000 replicate trees were considered significant (indicated at the nodes of corresponding branches). Results show that (a) the first DEP domain (DEP 1) of DEPDC6 clustered with the second DEP domain (DEP2) of P-Rex2 (also known as DEPDC2) (bootstrap 90%), and (b) the DEP2 domain of DEPDC6 clustered with Dishevelled 1 (DVL1) (bootstrap 86%) (FIG. 5a). Note that the DEPDC6 architecture mimics a truncated form of P-Rex2 (FIG. 5a), and the PDZ domain of DEPDC6 shares a 53-56% amino acid similarity with the tandem PDZ domains of P-Rex2. According to past reports, P-Rex2 is regulated by phosphatidylinositol (3,4,5)-trisphosphate (PIP3) and the βγ subunits of heterotrimeric G proteins.

Furthermore, P-Rex2 serves as a link between Rac activation and the PI3K pathway, and its tandem DEP domains interact directly with the carboxyl-terminal region of mammalian target of rapamycin (mTOR). We therefore performed a pull-down assay to test whether DEPDC6 also interacts with mTOR. HEK293T cells were transfected with pHA-DEPDC6 and harvested for co-immunoprecipitation experiments. Cell lysates were incubated with anti-HA antibodies and precipitated using protein A/G beads. Immunoprecipitants were resolved on SDS-polyacrylamide gels and analyzed by immunoblotting. A specific interaction was observed between endogenous mTOR and DEPDC6, but not in the control experiment. As shown in FIG. 5b, endogenous mTOR from HEK293T cells transfected with plasmid expressing HA-tagged DEPDC6 (HA-DEPDC6) was immuno-precipitated by anti-HA antibodies.

mTOR is a highly conserved protein kinase located downstream of PI3K and Akt. Its activation is associated with cell growth, survival, and proliferation. mTOR is found in two functionally and structurally distinct complexes, labeled mTOR complex 1 (mTORC1) and mTOR complex 2 (mTORC2). The dysregulation of mTORC 1 signaling is frequently observed in human cancer tissues. In HEK293T cells, we observed that the transient overexpression of DEPDC6 blocked the phosphorylation of ribosomal protein S6—the downstream target of the mTOR signaling pathway (FIG. 5c, lane 2). In addition, DEPDC6 overexpression slightly enhanced Akt^{S473} phosphorylation (FIG. 5c, lane 2). These results are consistent with a recent description of DEPDC6 as an mTOR-binding protein, given the name DEPTOR. Regarding the role of GNMT in the mTOR pathway, we found that GNMT can almost completely eliminate the suppressive effect of DEPDC6 on S6 phosphorylation (FIG. 5c, lane 5); in comparison, P-Rex2 has a much weaker suppressive effect (FIG. 5c, lane 6). According to these results, GNMT counteracts the inhibitory effects of DEPDC6 in the Akt/mTORC1 pathway. Further studies are needed to determine whether or not GNMT, DEPDC6, and mTOR are present in the same complex.

Since the DEP2 domain of DEPDC6 shares high sequence homology with DVL 1, we used a T-cell factor/lymphoid enhancer factor (Tcf/Lef) luciferase reporter assay to determine the effects of DEPDC6 on the Wnt/β-catenin pathway. HEK293A cells were co-transfected with TOPFLASH reporter (950 ng), pRL-TK vector (50 ng, to control for transfection efficiency), and pHA-DEPDC6 (50, 200, and 800 ng). pcDNA3 plasmid was added to make total DNA amounts equal. At 36 h post-transfection, cells were serum-starved for 24 h prior to treatment with L-Wnt3a CM for 6 h. Cell lysates were measured for luciferase activity, expressed as arbitrary units relative to the activity observed in unstimulated cells normalized for Renilla luciferase activity. Experiments were performed in triplicate; error bars represent S.D. According to our results, reporter activity induced by L-Wnt3a conditioned medium (CM) was dose-dependently downregulated by DEPDC6 (FIG. 5d).

A further experiment was preformed to find out the effects of DEPDC6 when the pathway was activated via β-catenin overexpression, HEK293A cells were co-transfected with TOPFLASH reporter (950 ng), pRL-TK vector (50 ng, to control for transfection efficiency), pHA-(3-catenin (50 ng), and pHA-DEPDC6 (50, 200 and 800 ng). pcDNA3 plasmid was added to establish equal amounts of DNA. Cells were harvested at 36 h post-transfection for luciferase activity measurements. In contrast to the results observed in L-Wnt3a treatment, as shown in FIG. 5e, DEPDC6 significantly enhanced reporter activity dose-responsively. A possible explanation for these results is by the linkage between Akt and Wnt/β-catenin pathways. GSK3β (a principle substrate of Akt) has been shown to phosphorylate β-catenin and facilitate its ubiquitination as well as proteasome-mediated degradation. Also, previous studies indicate that Ser9 phosphorylation by Akt leads to GSK3β inhibition (Cross, D. A. E., et al., Nature 1995; 378:785-789). Even though IGF-1 by itself does not allow the accumulation of free β-catenin to the threshold required to initiate transcription activation, the combination of IGF-1 and lithium chloride (a GSK3 inhibitor) does enhance β-catenin transcriptional activity. Accordingly, β-catenin overexpression may cooperate with DEPDC6 to enhance downstream transcriptional activity.

The Wnt signaling pathway plays an important role in embryonic development, regulating cell proliferation and cellular polarity and determining cell fate 19. When the Wnt ligand binds to Frizzled receptor its signals are transduced to DVL, followed by the inactivation of β-catenin degradation complexes. The stabilization of β-catenin supports its association with T-cell factor (Tcf) in cell nuclei and activates targeted genes. According to the observations described above, DEPDC6 is significantly up-regulated in human HCC (FIG. 4a, b), DEPDC6 down-regulates both the mTOR and Wnt signaling pathways, and GNMT is capable of counteracting the suppressive effects of DEPDC6 on the mTOR pathway.

Since the DEP2 domain of DEPDC6 shares high sequence homology with DVL1, we used a T-cell factor/lymphoid enhancer factor (Tcf/Lef) luciferase reporter assay to determine the effects of DEPDC6 on the Wnt/β-catenin pathway. According to our results, reporter activity induced by L-Wnt3a conditioned medium (CM) was dose-dependently downregulated by DEPDC6 (FIG. 5d). In contrast, when we activated the pathway via β-catenin overexpression, DEPDC6 significantly enhanced reporter activity dose-responsively (FIG. 5e). A possible explanation for these results is by the linkage between Akt and Wnt/β-catenin pathways. GSK3β (a principle substrate of Akt20) has been shown to phosphorylate β-catenin and facilitate its ubiquitination as well as proteasome-mediated degradation. Also, previous studies indicate that Ser9 phosphorylation by Akt leads to GSK3β inhibition. Even though IGF-1 by itself does not allow the accumulation of free β-catenin to the threshold required to initiate transcription activation, the combination of IGF-1 and lithium chloride (a GSK3β inhibitor) does enhance β-catenin transcriptional activity. Accordingly, β-catenin overexpression may cooperate with DEPDC6 to enhance downstream transcriptional activity.

More intriguing, our data indicate that DEPDC6 may enhance β-catenin-stimulated Tcf/Lef-dependent transcription activity via crosstalk between Akt and the Wnt pathway. It has been demonstrated that deregulation of Wnt pathway is implicated in the pathogenesis of liver cancer and mutation of components within β-catenin destruction complex, such as APC and AXIN, are common in many types of cancer including HCC. We previously used a Tcf/Lef reporter assay to demonstrate that (a) GNMT inhibits Wnt signaling, and (b) cyclin D1 and c-myc (target genes of Wnt signaling) are up-regulated in the adult livers and HCC tissues of GNMT −/− mice, meaning that both GNMT and DEPDC6 play important roles in PI3K/Akt/mTOR and Wnt/β-catenin pathways.

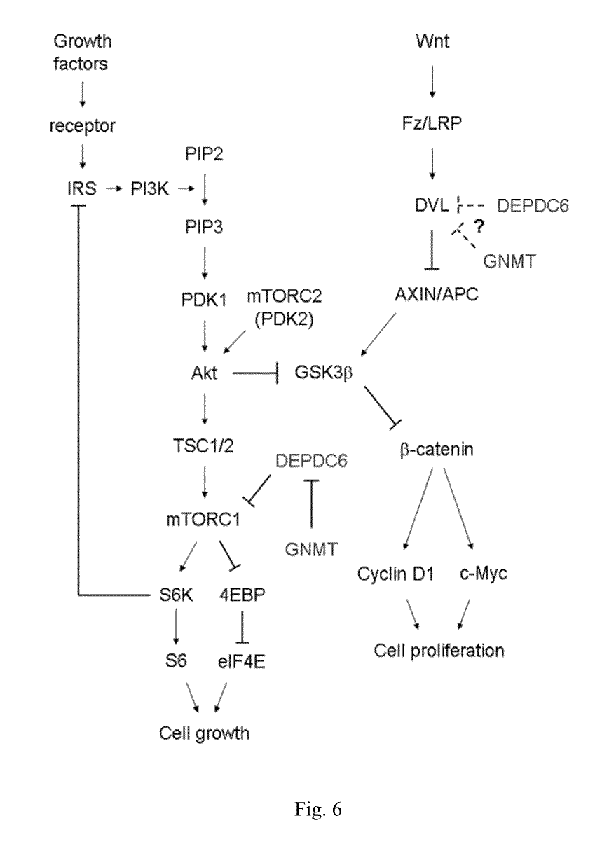

In the model shown in FIG. 6, growth factors promote mTOR-dependent signaling via PI3K/Akt. S6K inhibits insulin receptor substrate (IRS) by direct phosphorylation (resulting in a negative feedback loop) and is responsible for the Akt inhibition caused by high mTORC 1 activity. In some cancer cell lines, Rapamycin (an mTORC 1 inhibitor) treatment led to strong Akt activation due to loss of the negative feedback loop. A similar phenomenon was observed in the presence of DEPDC6; the DEPDC6 inhibitory effect was counteracted by GNMT overexpression. The precise relationship of DEPDC6 and GNMT to Wnt ligand-stimulated signaling remains unclear. DEPDC6 may enhance p-catenin-induced transcriptional activity by increasing Akt activity via negative feedback loop inhibition. The functions of GNMT and DEPDC6 in these pathways may provide valuable information for drug development and cancer prevention.

### Example 5

### Tissue Distribution of DEPDC6

Additionally, we detected the expression of DEPDC6 in various organs by IHC staining. Monoclonal Ab-D5A was used to detect expression profile of DEPDC6 in different organs. Multiple cancer tissue array (US Biomax, Cat. No. BCN721) was incubated with monoclonal antibody D5A. Signals were visualized using SuperPicTure™ Polymer Detection kits (Zymed, Invitrogen, Carlsbad, Calif., USA).

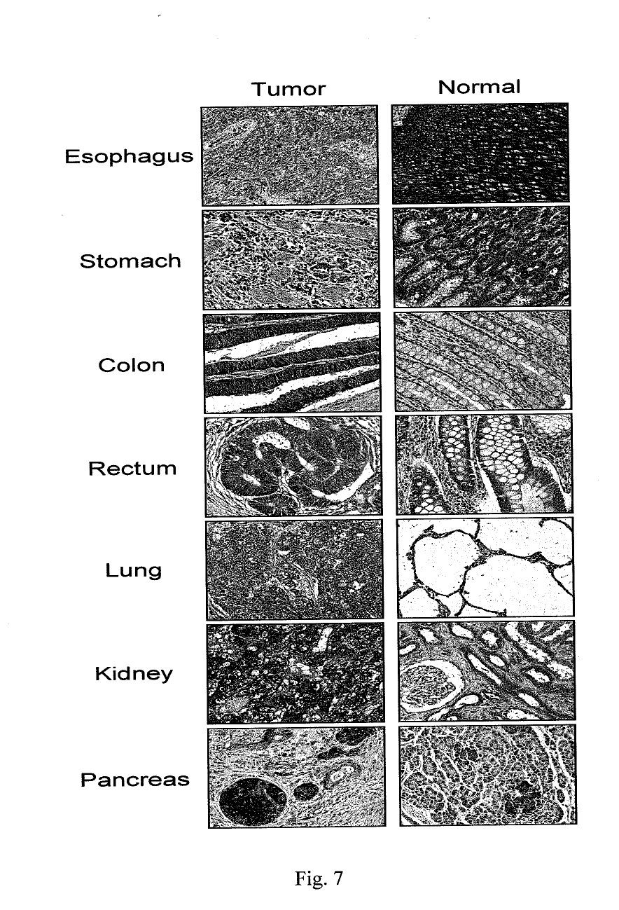

As shown in FIG. 7, DEPDC6 was expressed in the normal esophagus, stomach, rectum, lung, uterine cervix, breast, ovary and prostate. However, there was no DEPDC6 expression in normal colon epithelium. In kidney, DEPDC6 expressed both in distal and proximal convolution ducts but not in the glomerulus. In pancreas, DEPDC6 was detected in islet but not in exocrine cells. A comparison between these normal tissues to their corresponding tumor tissues indicated that DEPDC6 was up-regulated in colon and breast cancers (see, FIG. 7 and Table 5). DEPDC6 was highly expressed in tumor tissues from rectum, kidney and pancreas. By contrast, DEPDC6 was down-regulated in esophagus, stomach, uterine cervix and ovary cancers (FIG. 7 and Table 5). These results indicated that the role of DEPDC6 was varied in different organs and may be useful in diagnostic of different kinds of cancers.

TABLE 5
Expression profile of DEPDC6 in different
organ and their corresponding tumor tissue

DEPDC6 expression*

| | N (normal | T (tumor |
|---|---|---|
| Organ | tissue) | tissue) |
| Esophagus | ++ | + |
| Stomach | ++ | + |
| Colon | − | + |
| Rectum | + | + |
| Lung | + | + |
| Breast | −/+ | ++ |
| Uterine cervix | ++ | + |
| Ovary | + | − |
| Prostate | + | + |
| Kidney** | Ducts: + | ++ |
| | Glomerulus: − | |
| Pancreas*** | Exo: − | ++ |
| | Endo: + | |

*++, strong positive staining; +, positive staining; −/+, weak positive staining; −, negative staining
**Ducts: represent the distal and proximal ducts
***Exo, exocrine cells; endo, endocrine cells (islet)

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A DEP domain containing 6 (DEPDC6) protein identified as a tumor marker, which comprises the amino acid sequence described in SEQ ID NO: 1.

2. The method of claim 1, wherein the DEPDC6 protein is a GNMT binding protein.

3. A monoclonal antibody against the DEPDC6 protein of claim 1.

4. The monoclonal antibody of claim 3, which is anti-DEPDC6 monoclonal antibody D5 produced by hybridoma with as Deposit No. DSM ACC3180.

5. The monoclonal antibody of claim 3, which is used in the detection of DEPDC6 protein.

6. The monoclonal antibody of claim 3, which is used to detect the expression level of DEPDC6 protein in a human tissue.

7. The monoclonal antibody of claim 3, wherein the human tissue is isolated from an organ selected from the group of liver, colon, breast, rectum, kidney, pancreas and stomach.

8. A method for detecting the presence or the expression level of DEPDC6 protein in a tissue sample, which comprises incubating the tissue sample with the monoclonal antibody of claim 3, and analyzing the binding of the monoclonal antibody and DEPDC6 protein in the tissue sample.

9. The method of claim 8, wherein the analyzing method is ELISA.

10. The method of claim 8, wherein the analyzing method is immunohistochemical staining

11. The method of claim 8, the tissue sample is isolated from a tumorous organ.

12. The method of claim 9, wherein the organ is selected from the group of liver, colon, breast, rectum, kidney, pancreas and stomach.

13. A method for diagnostic a possible tumor in human, which comprises determining the presence or the expression level of DEPDC6 protein in a tissue sample isolated from the human subject.

14. The method of claim 13, wherein the presence or the expression level of DEPDC6 protein is determined by using the monoclonal antibody against of claim 3.

15. The method of claim 13, wherein the presence or the expression level of DEPDC6 protein is determined by real-time PCR.

16. A diagnostic kit for human cancer, which comprises the monoclonal antibody of claim 3.

17. The diagnostic kit of claim 16, wherein the human cancer is selected from the group of liver cancer, colon cancer, breast cancer, and stomach cancer.


SEQUENCE LISTING (9 sequences)

SEQ ID NO: 1 - protein, 409 aa, Homo sapiens
MEEGGSTGSAGSDSSTSGSGGAQQRELERMAEVLVTGEQLRLRLHEEKVIKDRRHHLKTYPNCFVAKELIDWLIEHKEASDRETAIKLMQKLADRGIIHHVCDEHKEFDNEVKAFMRGQRLYKDVKLFYRFRKDDGTFPLEKLMSPENTLLQPREEEGVKYERTFMASEFLDWLVQEGEATTRKEAEQLCHRLMEHGIIQHVSSKHPFVDSNLLYQFRMNFRRRRRLMELLNEKSPSSQETHDSPFCLGKQSHDNRKSTSFMSVSFSKEIKIVSAVRRSSMSSCGSSGYFSSSPTLSSSPPVLCNPKSVLKRPVTSEELLTPGAPYARKTFTIVGDAVGWGFVVRGSKPCHIQAVDPSGPAAAAGMKVCQFVVSVNGLNVLHVDYRTVSNLILTGPRTIVMEVMEELEC

SEQ ID NO: 2 - DNA, 30 bp, Artificial Sequence
  JSRV Env 1845 bp_R_Primer
cttaagctttcacgggtcgtcccccgcatc

SEQ ID NO: 3 - DNA, 24 bp, Artificial Sequence
  JSRV Env 252-725 bp Region_F-Primer
gcgtacattcctgatccgccaatg

SEQ ID NO: 4 - DNA, 24 bp, Artificial Sequence
  JSRV Env 252-725 bp Region_R_Primer
cggatgctgtcctcgatattcagg

SEQ ID NO: 5 - DNA, 22 bp, Artificial Sequence
  JSRV Env 481-502 bp_F_Primer
cgagtgactatctcaggcattg

SEQ ID NO: 6 - DNA, 20 bp, Artificial Sequence
  JSRV Env 522-541 bp_R_Primer
tagtatgcccttgcctagac

SEQ ID NO: 7 - DNA, 8 bp, Artificial Sequence
  Roch Universsal Probe set No. 48
actgggaa

SEQ ID NO: 8 - DNA, 1848 bp, Jaagsiekte sheep retrovirus (JSRV)
atgccgaagcgccgcgctggattccggaaaggctggtacgcgcggcagaggaactccctgacgcatcaaatgcaacgcatgacgctgagcgagcccacgagtgagctgcccacccagaggcaaattgaagcgctaatgcgctacgcctggaatgaggcacatgtacaacctccggtgacacctactaacatcttgatcatgttattattattgttacagcgggtacaaaatggggcagctgcggctttttgggcgtacattcctgatccgccaatgattcaatccttaggatgggatagagaaatagtacccgtatatgttaatgatacgagccttttaggaggaaaatcagatattcacatttcccctcagcaagcaaatatctctttttatggccttaccactcaatatcccatgtgcttttcttatcaatcgcagcatcctcattgtatacaggtatcagctgacatatcatatcctcgagtgactatctcaggcattgatgaaaaaactgggaaaaaatcatacgggaacggatctggacccctcgacattccgttttgtgacaagcatttaagcattggcataggcatagacactccttggactttatgtcgagcccgggtcgcatcagtatataacatcaataatgccaatgccacctttttatgggattgggcacctggaggaacacctgattttcctgaatatcgaggacagcatccgcctattttctctgtaaataccgctccaatataccaaacggaactatggaaacttttggctgcttttggtcatggcaatagtttatatttacagcccaatatcagtggaagcaaatatggtgatgtaggagttacaggatttttatatcctcgagcttgcgtgccgtatccattcatgttgatacaaggccatatggaaataacactgtcattaaatatttatcatttgaattgttctaactgcatactgactaattgtattaggggagtagccaaaggagaacaggttataatagtaaaacagcctgcctttgtaatgctgcccgttgaaatagctgaagcctggtatgatgaaactgctttagaattattacaacgcattaatacggctctcagccgccctaagagaggcctgagcctgattattttgggtatagtatctttaatcaccctcatagctacagctgttacggcttccgtatctttagcacagtctattcaagctgcgcacacggtagactccttatcatataatgttactaaagtgatggggacccaagaagatattgataaaaaaatagaagataggctatcagctctatatgatgtagtcagagtcttaggagagcaagttcagagcattaattttcgcatgaaaatccaatgtcatgctaactataaatggatttgtgttacaaaaaagccatacaatacttctgattttccatgggacaaagtgaagaaacatttgcaaggaatttggttcaatactaatctatcgttagaccttttacaactgcataatgagattcttgatattgaaaattcgccgaaggctacactaaatatagccgatactgttgataatttcttgcaaaatttattctctaattttcctagtctccattcgctgtggaaaaccctgattggtgtaggaatacttgtgtttattataattgtcgtaatccttatatttccttgccttgttcgtggcatggttcgcgattttctaaagatgagagttgaaatgctgcatatgaaatatagaaatatgttacagcaccaacatcttatggagcttttaaaaaataaagagaggggagatgcgggggacgacccgtga

SEQ ID NO: 9 - DNA, 3792 bp, Homo sapiens
aagcttgaagactgctgctctctaccacgttagctcccctgtggcggaggatgactgtcctaagagctcataggcacggggcaaggggagcctggctgtcagctgcctgggcttctagtcttgtgctttttgcacaccagtccagggaacgaagaccactggctttaagatgcttccccagctgtccccagactctgccagcaggggattctctggtctgagcttaagttgtgttctcccagccagggatgcccctgccctttgatgtctccttgctgccacacatttagccgccctccccatgccagcttggggggagggaagcagtgagggtagggaggtggctggggcagctgggcaactgtccccacccgtccctggcacggctctgcccagtacacaaagagcaaagtgaatcttgtccccacccctgcagctgaggggctggaggaggaaacggggaggcccacacagaaggggtggccaccgtggggctgtccatcactcagggctctcagagggagtcaacccagaaacagacaaagagggtgagtctgggctgtgttcttagctagtgagaggtcccctagaggatgaagtagatgatgctaatgaggatgactggatgtcacacccatgatgctattaggtcctcataatagcatagtgaggtggacagctagtacctgacccatctcacagatgaacatactaatgcctaacaaagcagaacgactcacgctgggtcccagagctggccagtggaagcactgagacctccacatactgaaggcatggactattgaccgctgttggtattggtctcatcattgactatcattaagtgttggctgtttgcatgcttcctgcccagtggcaggttcaaagaagcccgcaggaagcgtgctcctttctttcccagggcccgcaattgggctggaagatagagcaacaaaaagcgcccatgtaactcatgggaacattcatgtgtgctgaatggcaggtgaaggtgccacagagaggctgaggatttcagagggcaccatgaactggagtgaggtcgcagagcaggtgccattggctcttggcctgtttgggtgggtggcattcagagaggtggaaggtcagatgcactgttcacgcctgtaatctcagcactttggaaggccaaggtaggaggatcacacgaggccaggagatcaacgctgcagtgagctatgaagctgtgattgcaccactgcactccagcttgggtaacagagtgagaccctgtctctaaataattaaataaataaaataaaaataaaaccggagaagtggagagggattggaggtgggctttcacagagggagaaacggcttaagtacaggccaaaaagtgagaaggctgcagacagggctggtagggggagggggaagtttggcacacccagctaaaggtccttctgtgtctccttctccaaggaacccaagaccttcacttggttggtgtgagcactccaggaggcaggcaccctccctcagccctcaagcaagcaaaaatgggtttaaaaaaagaaggagaagaagcagcagcagcagccgccacagagcttgtgacagctacagcctaagggcaacaggcaggggagaccaaggaccagaaagagcagaggctttttcaaagaaagagatccctctcccagcacccagcgatggcggcaaaccccacccacagtgcctgctaagaacaagtcccacgtgagaacaacatggccccccgagatgcccacagggaccccgagatgcctgcagtgcttggctctcccgctggccagctgcccacctggctcaggcccagtactcgtgagtcagccgatcaatcccaatgttgccaggatgatgggggcgggagtaagggccctgggggagggcaggggtgggcactgcaggcgagctgtctcccacatctggcacctgcacacagctgaggccgagctgagaggatgcttctgtgggctccccctcctcccggcacccctcccctcctttcactgtcctaggacactctctggctgctggagtcttaggcaaatatttaaaggggcagcaagggggtgaggagggtggtgggagcaaacacttccctccctttcttcctccctgcgcttctcaggggctctcagttcagatgccatgctgttatgcaaccttggggctgaaggccctccagatggagagggggacaggggaacctgccagctcatgaccgaagggcagggcccaggtgggagggggctggggcaggggacaggaactggggtggcatgttaaaggacaggaggctggttgggcatggtggctcacacctgtaatcctagcactttaggaggccgagatcacttgagcccaggagttcaagaccagcctgggcaacatggtgaaaactcatctctataaaacaagcaaaaattagctgggcacaatggcatgcactggtagtcccagctacttgggatgctgaggtgtgaggatcaccggagtccaggaggtcaacgctgcagtgagcagtgatctcgctactgcatgccagcctgggtgataaagtgagaccctgtctcacaacaaaacaaaacaaaacaaaacaaaaggataggaggttaagggagcgagcccaggcctggactctgccacagtcacctgagtttagaggtgaagggactttagagaccacctggcccaaggagtgaaagggaacctgagagaaagggtgagccagcccaaagtcattggcagatttgacctcgtaaatacatagagatggctttgggaaggcactaggaaagacagagaaaagagaaggagacagtcctcaaagctgatcgtatttgggtgagtattattctcagggcaaatttaggatcagaggatgcagaaaggggagtctagaggggtagagtgtagaccacagggtgagtgagctgattcgaggatggggagactgggagcccaccagtgaccagagccagccctgttcagggctgtccgggcagaagaaagcagtgtcagacctggaatctgccatcagcacagcctgcaattgacagacaagcccagagcaaagaaggaagcactgcacatgagtaagagcttgccaccagtggggacagagtttccagaattaggaaaataatcactgggggcaagtttgaggttggtaccagatatgtgggaggaggcaaggtaagggaaagagtacttgaagttggaactggtccttgcagggaaatgcacatttatgaaaccccgaaaactgatgtcaaagcacctcctgccttgggcagagtcctctcagagtctacaggtgctgcctccagaaccctcttcctggagcgcatccctatgtatctagaaattctgctgggaaatatgatggtcagacccttggccacctgaaaggttcagggtggtagaagaaaaaggaaagccacagggcagcaggggcaggtgccagcaaggaaggcaggcacgccaggaagacacccatggtgagaagtgcagatggcccgagggcaagtttgctcaactcacccaggtttgctcttgctggggccaagaggactcatgtgccagggccaagggcccttgggggctctcacagggggcttatctgggcttcggttctggagggccaggaacaaacaggcttcaaagccaagggcttggctggcacacagggggcttggtccttcacctctgtcccctctccctacggacacatataagaccctggtcacacctgggagaggagga